(19) United States
(12) Patent Application Publication
(10) Pub. No.: US 2011/0124574 A1
(43) Pub. Date: May 26, 2011

(54) RESTORATION OF ESTROGEN RECEPTOR-(ALPHA) ACTIVITY

(76) Inventors: Tommy Andersson, Malmo (SE); Caroline Elizabeth Ford, Randwick (AU)

(21) Appl. No.: 12/990,253

(22) PCT Filed: Apr. 30, 2009
(86) PCT No.: PCT/SE09/50475

§ 371 Date: Jan. 24, 2011

(30) Foreign Application Priority Data
Apr. 30, 2008 (SE) 0800977-1

(51) Int. Cl. A61K 38/08; A61K 38/10; A61P 35/00

(52) U.S. Cl. 514/19.4

(57) ABSTRACT

One third of all breast cancers are estrogen receptor alpha (ERα) negative, have a poor overall prognosis and do not respond well to currently available endocrine therapies. Use of a Wnt5-α protein or a peptide thereof, such as a recombinant Wnt-5a protein or a Wnt-5a derived hexapeptide (Foxy-5) possessing Wnt-5a signaling properties, enables restoration of ERα expression and makes it possible to treat such breast cancers with selective estrogen receptor modulators, such as tamoxifen, or aromatase inhibitors.

### TECHNICAL FIELD

The present invention relates to establishment or restoration of estrogen receptor-α expression and activity, and thereby of sensitivity to estrogen receptor modulators, such as tamoxifen, in estrogen receptor alpha negative breast cancer cells.

### BACKGROUND OF THE INVENTION

Breast cancer remains one of the most common diseases of women worldwide. Despite advances in detection and treatment, in many patients the disease progresses to metastasis. Patients negative for the nuclear hormone receptor, estrogen receptor alpha (ERα) have a particularly poor prognosis (1). Analysis of a clinical cohort of breast cancer patients revealed a statistically significant association between loss of ERα expression and loss of Wnt-5a expression (2). It has been shown that loss of Wnt-5a expression in breast cancer occurs at the translational, and not the transcriptional level. Consequently, it was hypothesized that Wnt-5a might be capable of regulating ERα levels, and not the other way around. In the present work an investigation of such a relationship between these two key proteins in breast cancer has been established.

Wnt-5a is a member of the large family of Wnt molecules, and its altered expression has been associated with cancers including breast cancer, colon cancer, hepatocellular carcinoma and melanoma. In breast cancer, Wnt-5a has been shown to increase adhesion and reduce migration of epithelial cells explaining its link to the metastatic process and better patient outcome (2). A formylated hexapeptide, Foxy-5, capable of mimicking the effects of Wnt-5a on adhesion and migration of breast cancer cells has previously been developed. While it is unlikely that this peptide maintains all of the effects of Wnt-5a signaling, the inventors believe this peptide has a clear and immediate therapeutic potential. Peptides derived from Wnt5-a have been described earlier i.a. in WO 2006/130082 and WO 01/32708.

One factor contributing to the poor prognosis for ERα negative breast cancer patients is that endocrine therapies including treatment with tamoxifen, one of the major drugs used to treat breast cancer, are ineffective in ERα negative patients (1). Tamoxifen is referred to as a selective estrogen receptor modulator (SERM), as it acts as an agonist in some tissues, and an antagonist in other tissues. It is thought that tamoxifen works by binding to the ERα, causing a conformational change that prevents the recruitment of coactivators resulting in altered transcription of estrogen regulated genes and cell proliferation. Thus, in patients lacking ERα expression, tamoxifen is mostly ineffective. Endocrine therapies also includes treatment with aromatase inhibitors, such as anastrozole, exemestane or letrozole. However, treatment with aromatase inhibitors is mostly ineffective in patients lacking ERα expression, for the same reasons as discussed above for selective estrogen receptor modulators.

Therefore a new treatment approach for ERα negative breast cancer patients has been suggested; instead of developing brand new therapeutics to treat ERα negative patients, what if these patients could be sensitized to respond to currently effective, approved and widely available treatment regimes, such as tamoxifen? Such a shift in thinking is currently underway and it has been suggested that if the patients' expression of certain genes could be modified in order to upregulate ERα, these patients could be treated effectively again (3). Researchers have restored ERα expression in ERα negative breast cancer cells using transfection of the full length ERα plasmid, or treatment with DNA methyl transferase (DNMT) and histone deacetylase (HDAC) inhibitors, such as 5-aza-dC and Trichostatin A (3-6). However none of these strategies are feasible for direct clinical use.

### SUMMARY OF THE PRESENT INVENTION

In view of the fact that one third of all breast cancers are estrogen receptor alpha (ERα) negative, have a poor overall prognosis and do not respond well to currently available endocrine therapies, new treatment strategies are required. Thus it was investigated whether administration of recombinant Wnt-5a or the Wnt-5a derived hexapeptide, Foxy-5, to ERα negative breast cancer cells could upregulate their expression of ERα, and possibly render them responsive to selective estrogen receptor modulators or aromatase inhibitors. It was found that by reconstituting ERα expression by employing a natural cell surface receptor ligand, or a hexapeptide mimicking this ligand rendered breast cancer cells responsive to current endocrine treatment with a selective estrogen receptor modulator, such as tamoxifen, or an aromatase inhibitor, such as anastrozole, and thus suggest an important progress of clinical management of breast cancer. Concordant treatment with a Wnt-5a mimicking hexapeptide and currently available ERα modulators constitutes a novel and beneficial treatment strategy for breast cancer patients with ERα negative tumors.

One aspect of the invention thus relates to use of a Wnt5-α protein or a peptide thereof for the production of a pharmaceutical composition for use in treatment of a subtype of breast cancer characterized by lack of estrogen receptor-α activity.

A further aspect of the invention relates to a Wnt5-α protein or a peptide thereof for use in treatment of a subtype of breast cancer characterized by lack of estrogen receptor-α activity.

Yet another aspect of the invention relates to a Wnt5-α protein or a peptide thereof for use in treatment of breast cancer.

Another aspect of the invention relates to a Wnt5-α protein or a peptide thereof for use in treatment of breast cancer in an estrogen receptor-α negative patient.

A further aspect of the invention relates to a method for restoring estrogen receptor-α activity by administering a therapeutically active amount of Wnt5-α protein or a peptide thereof to a human lacking estrogen receptor-α for a time sufficient to induce such estrogen receptor-α activity by restoring such receptors.

Another aspect of the invention relates to a method for facilitating or enhancing endocrine post-treatment in a human suffering from breast cancer and lacking estrogen receptor-α activity, wherein a therapeutically effective amount of Wnt5-α protein of a peptide thereof is administered for a time sufficient to induce estrogen receptor-α activity.

### BRIEF DESCRIPTION OF THE DRAWINGS

FIG. 1: Basal expression of ERα, Frizzled 5, PR and Wnt-5a in experimental cell lines.

A: Protein lysates from cells grown in culture were analyzed via SDS-PAGE and Western blotting for proteins of interest. Tubulin expression was used as a loading control. B: RNA was extracted from cell lines and subjected to cDNA synthesis and RT-PCR for our genes of interest. The T47D human breast cancer cell line was used as a positive control for both protein and mRNA analysis as it is known to express all the genes of interest for our study. β-actin expression was used as a housekeeping gene. The negative control represents a water control.

FIG. 2: Wnt-5a signaling restores ERα expression.

Breast cancer cells were grown in 6 well plates, and stimulated with recombinant Wnt-5a protein (rW5a), the Wnt-5a derived Foxy-5 peptide (F5), recombinant Wnt-3a protein (rW3a), or a formylated random hexapeptide (Rdm) for 24 or 48 h. Following treatment cells were lysed and subjected to SDS-PAGE, transferred to nitrocellulose membranes and blotted for ERα expression. A: MDA-MB-231 cells stimulated with recombinant Wnt-5a, Foxy-5, Wnt-3a, Rdm. B: MDA-MB-468 cells stimulated with recombinant Wnt-5a or Foxy-5. C: 4T1 cells stimulated with recombinant Wnt-5a or Foxy-5. Positive controls (Pos) were included in order to confirm the correct band size for ERα. Two different positive controls were used: T47D cell lysates known to express ERα and MDA-MB-231 cells transiently transfected with a full length ERα plasmid, resulting in extremely high ERα expression (top row, right panel, third row, right panel).

FIG. 3: Wnt-5a signaling restores ERα transcription.

Breast cancer cells were grown in 6 well plates and stimulated with recombinant Wnt-5a protein (rW5a) or the Wnt-5a derived Foxy-5 peptide (F5), for 6, 12, 18 or 24 h. RNA was extracted at the end time point, cDNA synthesized and subjected to RT-PCR for ERα and the housekeeping gene, β-actin. A: MDA-MB-231 breast cancer cells, B: MDA-MB-468 breast cancer cells. The positive control (Pos) is RNA extracted from T47D cells which express ERα. The negative control represents a water control.

FIG. 4: Wnt-5a signaling demethylates the ERα promoter.

MDA-MB-231 cells were grown in normal media and either left untreated, or stimulated with rWnt-5a protein (rWnt-5a, 0.6 μg/ml)) or the Wnt-5a derived Foxy-5 peptide (F5, 100 μM), for 48 hours. MCF-7 cells were grown for the same amount of time, and were left untreated. DNA was extracted from each sample and subjected to bisulfite modification. Bisulfite treated DNA was subjected to bisulfite genomic sequencing (BGS) of the ERα promoter using nested PCR with primers for the ERα promoter region. PCR products were cloned and 10 random clones sequenced. Filled (black) circles represent methylation at a given cytosine, empty (white) circles represent either unmethyllated cytosine or cytosines demethylated following rWnt-5a or Foxy-5 treatment. The numbers represent the position of CpG dinculeotides relative to the transcription start site (+1). The TATA box is located between positions −17 and +13.

FIG. 5: ERα is active and capable of downstream transcription.

MDA-MB-231 cells were grown in 6 well plates and stimulated with recombinant Wnt-5a protein (rW5a) or the Wnt-5a derived Foxy-5 peptide (F5), for 24 or 48 h. A: Following treatment, cells were lysed and subjected to SDS-PAGE, transferred to nitrocellulose membranes and blotted for phospho-ERα expression. The positive control (Pos) represents cell lysates from T47D cells expressing ERα B and C: RNA was also extracted from stimulated cells and samples tested for progesterone receptor (PR) (B) and pS2 (C) mRNA using semi-nested RT-PCR. The positive control (Pos) is RNA extracted from T47D cells that express ERα. The negative control represents a water control. PCR results are representative of three separate experiments.

FIG. 6: Upregulation of ERα renders previously unresponsive breast cancer cells, sensitive to tamoxifen treatment.

MDA-MB-231 cells were grown in 6 well plates and stimulated with recombinant Wnt-5a protein (rW5a) or the Wnt-5a derived Foxy-5 peptide (F5), for 24 or 48 h. Cells were treated with tamoxifen for the final 20 h and their apoptotic responses were measured via different methods. A: Treated cells were stained with Hoechst to visually assess apoptotic cells displaying altered nuclear morphology per treatment. Arrows highlight apoptotic cells. Bars represent 10 μM. B: Following treatment with rW5a, F5 and tamoxifen, or tamoxifen alone, cells were lysed and subjected to SDS-PAGE, transferred to nitrocellulose membranes and blotted for cleaved caspase 3. C: Treated cells were assessed for their relative caspase 3 activity using fluorometric spectrophotometry. The graph represents 6 separate experiments. * P<0.01, ** P<0.001. D: MTT assays were also performed on MDA-MB-231 cells treated with rWnt-5a, F5 and tamoxifen, or tamoxifen alone (MDA-MB-231 and MCF-7 cells) to assess cell growth inhibition. The graph represents the average of 6 separate experiments, with standard deviation represented by error bars. ** P<0.01, *** P<0.001.

FIG. 7: The expression of genes directly regulated by ERα is lost following tamoxifen treatment.

MDA-MB-231 cells were grown in 6 well plates and stimulated with recombinant Wnt-5a (rW5a) or the Wnt-5a derived Foxy-5 peptide (F5), for 24 h or 48 h. The ERα ligand estradiol was added for the final 22 h, and tamoxifen for the final 20 h to a subset of samples. RNA was extracted at the end time point, cDNA synthesized and subjected to RT-PCR for Cathepsin D (CATD), ER-binding fragment associated antigen 9 (EBAG9) and the housekeeping gene, β-actin.

FIG. 8: Foxy-5 upregulates ERα in vivo.

2.5×10⁴ 4T1 breast cancer cells were inoculated into the mammary fat pads of 8-week old Balb/C mice. The animals were subsequently treated with either PBS alone, the Rdm control peptide (20 μg) or Foxy-5 (20 μg), every 4^{th} day, for 25 days. RNA was extracted from primary breast tumors from 4 animals in each group, and subjected to RT-PCR for murine ERα. Shown are primary tumor samples from two animals of each treatment group.

### DETAILED DESCRIPTION OF THE PRESENT INVENTION

In particular the present invention relates to the use of the Wnt5-α protein, such as a recombinant Wnt5-a protein, or a peptide thereof for enhancing or restorating estrogen receptor-α activity. This is of particular interest in treatment of breast cancer when the patient is estrogen receptor-α negative.

After enhancement or restoration of the estrogen receptor-α activity it is possible to use an endocrine treatment for the patient, such as treatment with a selective estrogen receptor modulator, such as tamoxifen or treatment with an aromatase inhibitor, such as anastrozole. When using selective estrogen receptor modulators, such as tamoxifen, or aromatase inhibitors for treatment of breast cancer the outcome is often unsuccesful in estrogen receptor-α negative patients, unless a Wnt5-α protein, such as a recombinant Wnt5-a protein, or a peptide thereof is used in accordance with the invention.

In a preferred embodiment thereof the Wnt5-α peptide is one or more having one of the following sequences:

| | |
|---|---|
| MDGCEL | SEQ. ID. NO. 1 |
| GMDGCEL | SEQ. ID. NO. 2 |
| EGMDGCEL | SEQ. ID. NO. 3 |
| SEGMDGCEL | SEQ. ID. NO. 4 |
| TSEGMDGCEL | SEQ. ID. NO. 5 |
| KTSEGMDGCEL | SEQ. ID. NO. 6 |
| NKTSEGMDGCEL | SEQ. ID. NO. 7 |
| CNKTSEGMDGCEL | SEQ. ID. NO. 8 |
| LCNKTSEGMDGCEL | SEQ. ID. NO. 9 |
| RLCNKTSEGMDGCEL | SEQ. ID. NO. 10 |
| GRLCNKTSEGMDGCEL | SEQ. ID. NO. 11 |
| QGRLCNKTSEGMDGCEL | SEQ. ID. NO. 12 |
| TQGRLCNKTSEGMDGCEL | SEQ. ID. NO. 13 |
| GTQGRLCNKTSEGMDGCEL | SEQ. ID. NO. 14 |
| LGTQGRLCNKTSEGMDGCEL | SEQ. ID. NO. 15 |

or a formylated derivative thereof. It is also possible to use a combination of two or more of these peptides.

### Methods

### Cell Culture

Five breast cancer cell lines were used in this study. MDA-MB-231, MDA-MB-468, MCF-7, T47-D and 4T1 cells were all obtained from the American Type Tissue Collection (ATCC), and grown according to ATTC recommendations. The 4T1 cells were grown in RPMI medium (R8758) supplemented with 10% Fetal Calf Serum (FCS), 1.5 g/L sodium bicarbonate, 10 mM HEPES, and 1 mM sodium pyruvate. The MDA-MB-231, MDA-MB-468, and MCF-7 cell lines were grown in DMEM with 10% FCS. All cell medium contained the addition of 5 U/ml penicillin, 0.5 U/ml streptomycin and 2 mM glutamine. Cells were also grown in “hormone free media” lacking phenol-red and supplemented with 5% charcoal treated FCS for some experiments, as indicated. All cells were incubated in a humidified chamber at 37° C. with 5% CO₂

Stimulation with Recombinant Wnt-5a, Recombinant Wnt-3a, Foxy-5 or a Formylated Random Peptide

Stimulation of cells was performed with recombinant Wnt-5a (0.6 μg/ml) and recombinant Wnt-3a (0.1 μg/ml and in a control experiment, 0.6 μg/ml) (R&D Systems Abington, UK) for times as indicated. The Wnt-5a derived formylated hexapeptide, Foxy-5 (formyl-MDGCEL) (SEQ. ID. NO. 1) designed in the laboratory of the inventors, and a formylated random hexapeptide (formyl-MSADVG) (SEQ: ID: NO: 16) were either synthesized by Pepscan Presto (Lelystad, The Netherlands) or Inbiolabs (Tallinn, Estonia). The peptides were purified by RP-HPLC and mass spectrometry, and the >95% pure peptides were synthesized three times. Cells were treated with Foxy-5 or random peptide at a concentration of 100 μM for times as indicated. All other chemicals if not otherwise stated were purchased from Sigma Chemicals (St. Louis, Mo.).

Peptides which are known to possess Wnt5-alpha activity are

| | |
|---|---|
| MDGCEL | SEQ. ID. NO. 1 |
| GMDGCEL | SEQ. ID. NO. 2 |
| EGMDGCEL | SEQ. ID. NO. 3 |
| SEGMDGCEL | SEQ. ID. NO. 4 |
| TSEGMDGCEL | SEQ. ID. NO. 5 |
| KTSEGMDGCEL | SEQ. ID. NO. 6 |
| NKTSEGMDGCEL | SEQ. ID. NO. 7 |
| CNKTSEGMDGCEL | SEQ. ID. NO. 8 |
| LCNKTSEGMDGCEL | SEQ. ID. NO. 9 |
| RLCNKTSEGMDGCEL | SEQ. ID. NO. 10 |
| GRLCNKTSEGMDGCEL | SEQ. ID. NO. 11 |
| QGRLCNKTSEGMDGCEL | SEQ. ID. NO. 12 |
| TQGRLCNKTSEGMDGCEL | SEQ. ID. NO. 13 |
| GTQGRLCNKTSEGMDGCEL | SEQ. ID. NO. 14 |
| LGTQGRLCNKTSEGMDGCEL | SEQ. ID. NO. 15 |

or a formylated derivative thereof. These peptides can be used alone or in a mixture of two or more.

### Cell Lysis and Western Blot Analysis

Cells were lysed in Triton lysis buffer (50 mM Tris (pH7.5), 1% Triton x-100, 140 mM NaCl, 0.5 mM EDTA, 0.5 MgCl₂, 10 mM NaF) with the addition of fresh leupeptin (1 μg/ml), Pefabloc (2 mM), aprotinin (20 μg/ml) and Na₃VO₄ (4 mM). Lysates were incubated on ice for 15 minutes, then pre-cleared by centrifugation for 10 minutes at 8000 g. Cell lysates were separated according to size on 8-12% SDS-polyacrylamide gels and subsequently electrically transferred to PVDF or nitrocellulose membranes. Membranes were blocked for 1 h at room temperature in TBS-Tween (0.01%) with 5% milk. Membranes were incubated with primary antibodies overnight at 4° C. in TBS-Tween (0.01%) with 3% milk, then washed 3 times for 10 minutes in TBS-Tween (0.01%). Visualization of proteins was performed via the addition of a secondary antibody conjugated to horse radish peroxidase to the membrane which was then incubated for 1 h at room temperature in TBS-Tween (0.01%) with 3% milk. Membranes were washed 3 times for 10 minutes in TBS-Tween (0.01%) and then incubated in ECL and developed with hyperfilm. Scanning and densitometry was performed using a Bio-Rad (Hercules Calif.) GS-800 densitometer with Quantity One software.

### Antibodies

Antibodies were used at the following dilutions: Estrogen Receptor α: HC-20 (Santa Cruz Biotechnology) 1:1000, Wnt-5a: Antibody developed in our laboratory against a Wnt-5a sequence with 100% homology between human and mouse 1:1000 (2), Progesterone Receptor: 6A1, Detects both A and B isoforms (Cell Signaling Technology), Frizzled 5: (Upstate) 1:1000, Cleaved Caspase 3: Asp175 (Cell Signaling Technology) 1:1000, Phospho-ERα (Ser 118): 16J4 (Cell Signaling Technology) 1:1000, Tubulin: DM1A (Santa Cruz Biotechnology) 1:10000. All secondary antibodies were from Dako Chemicals and were used at the following dilutions: Goat anti rabbit 1:10000, Goat anti mouse 1:7500, Rabbit anti goat 1:7500.

### RNA Extraction

RNA extraction was performed in a designated clean RNA area with the addition of 500 μl TRIzol to each sample. 100 μl of chloroform was then added and samples centrifuged at 4° C. at 250 g for 10 min. 250 μl of isopropanol was added to the clear upper phase and samples centrifuged for 15 min at 4° C. at 16000 g. The supernatant was removed and the pellet was washed in 75% ethanol and resuspended in DEPC treated water. RNA was treated with DNase 1 (Sigma) at 37° C. The RNA concentration was measured using a Nanodrop Spectrophotometer ND-1000 (Bio-Rad (Hercules Calif.)).

cDNA Synthesis & Reverse Transcriptase PCR (RT-PCR)

cDNA was synthesized from 1 μg of total RNA using M-MuLV reverse transcriptase (Fermentas) in a MJ Mini Personal Thermal Cycler (Bio-Rad (Hercules Calif.)). All RT-PCR was performed in a designated clean PCR hood. RT-PCR was performed using a master mix containing with 5 μl of 10× buffer, 5 μl of 25 mM MgCl₂, 1 μl 10 mM dNTP, 1 μl forward primer, 1 μl reverse primer and 0.2 μl of Taq polymerase (Fermentas (Ontario, Canada)) per sample. For detection of the progesterone receptor (PR), semi nested RT-PCR was performed to increase sensitivity, whereby 10 μl of the initial PCR reaction product was added to a second PCR reaction with a second internal reverse primer. Both A and β isoforms are amplified with these primers.

Primer sequences were as follows.

(SEQ. ID. NO. 17)
ERα forward:
5′ CAC CCT GAA GTC TCT GGA AG 3′,
(SEQ. ID. NO. 18)
ERα reverse:
5′ GGC TAA AGT GGT GCA TGA TG 3′,
(SEQ. ID. NO. 19)
Cathepsin D forward:
5′ GTA CAT GAT CCC CTG TGA GAA GGT 3′,
(SEQ. ID. NO. 20)
Cathepsin D reverse:
5′ GGG ACA GCT TGT AGC CTT TC 3′,
(SEQ. ID. NO. 21)
EBAG9 forward:
5′ GAT GCA CCC ACC AGT GTA AAG A 3′
(SEQ. ID. NO. 22)
EBAG9 reverse:
5′ AAT CAG GTT CCA TTG TTC CAA AG 3′,
(SEQ. ID. NO. 23)
β Actin forward:
5′ TTC AAC ACC CCA GCC ATG TA 3′
(SEQ. ID. NO. 24)
β Actin reverse:
5′ TTG CCA ATG GTG ATG ACC TG 3′
(SEQ. ID. NO. 25)
Wnt-5a forward:
5′ GGA TTG TTA AAC TCA ACT CTC 3′,
(SEQ. ID. NO. 26)
Wnt-5a reverse:
5′ ACA CCT CTT TCC AAA CAG GCC 3′
(SEQ. ID. NO. 27)
PR forward:
5′ TCA TTA CCT CAG AAG ATT TAT TTA ATC 3′,
(SEQ. ID. NO. 28)
PR reverse 1:
5′ ATT GAA CTT TTT AAA TTT TCG ACC TC 3′,
(SEQ. ID. NO. 29)
PR reverse 2:
5′ATT TTA TCA ACG ATG CAG TCA TTT C 3′.

All RT-PCRs were performed at least 3 times, and controls lacking reverse transcriptase were routinely included to rule out DNA contamination.

### Nuclear Staining for Analysis of Apoptotic Cells

MDA-MB-231 cells were plated on cover slips and allowed to adhere. Wnt-5a (0.6 μg/ml) or Foxy-5 (100 μM) were added for 24 or 48 hours. Cells were then treated with tamoxifen (5 μM) for the last 20 hours. MCF-7 cells were used as a positive control. The cells were fixed for 15 min in ice cold paraformaldehyde (4%), washed and incubated in the dark with 10 μg/ml Hoechst 33342 stain (Invitrogen) for 10 minutes. The cells were washed with PBS and mounted with Dako Cytomation fluorescent mounting medium. The morphology was analyzed with Nikon E800 Eclipse Microscope with 60× objective.

### DNA Extraction & Bisulfite Genomic Sequencing (BGS)

DNA was extracted from cells according to standard procedures. 1 μg of DNA was then bisulfite treated using the EpiTect Bisulfite Kit (Qiagen) and amplified via nested PCR with primers for the ERα promoter region. PCR products were cloned using the TOPO TA cloning kit (Invitrogen). 10 random clones were sequenced using an AB13730 DNA analyzer (Applied Biosystems).

### Caspase 3 Activity Assay

Caspase 3 activity was determined via fluorescent spectrometry. The fluorogenic peptide DEVD-amc (Upstate Biotech) was used as a substrate. MDA-MB-231 cells were grown in 6 well plates and stimulated with recombinant Wnt-5a protein (0.6 μg/ml) or Foxy-5 peptide (100 μM) for 24 or 48 hours. Cells were then treated with tamoxifen (Sigma) at a concentration of 1 μM for 20 hours. Floating and adherent cells were lysed in caspase lysis buffer (10 mM Tris-HCl, 10 mM NaH₂PO₄/Na₂HPO₄, 130 mM NaCl, 1% Triton-X-100, 10 mM NaPPi), and 50 μl triplicates added to the reaction wells with 200 μl HEPES buffer and 3 μl of DEVD-amc. Reactions were incubated at 37° C. for 1 h and analysed on a FLUOstar plate reader (BMG Lab technologies). The total protein content of each lysates was measured using the Coomassie Plus Protein Assay and read outs averaged and adjusted accordingly. The experiment was performed 6 times, and results averaged.

### MTT Proliferation Assay

Cell proliferation was measured via MTT assay (Vybrant) following manufacturers instructions. Briefly, MDA-MB-231 and MCF-7 cells were grown in 96 well plates, then either left unstimulated, or stimulated with rWnt-5a (0.6 μg/ml) or Foxy-5 peptide (100 μM) for 24 or 48 hours. Cells were then treated with 5 μM tamoxifen (Sigma) for the final 20 hours. All cells were then labeled with MTT (3-(4,5-dimethylthiazol-2-yl)-2,5-diphenyltetrazolium bromide), incubated at 37° C. for 4 hours and absorbance measured on a Biorad 680 microplate reader (Biorad). The raw absorbance was measured in 9 replicates at 570 nm, and read outs averaged and adjusted accordingly. The experiment was performed 6 times, and results averaged.

### In Vivo Studies

2.5×10⁴ 4T1 breast cancer cells were inoculated into the mammary fat pads of 8 week old Balb/C mice, that were subsequently treated with either PBS alone, the Rdm control peptide (20 μg), or Foxy-5 (20 μg) every 4^{th} day, for 25 days as described in a previous publication from the inventors (9). RNA was extracted from flash frozen primary breast tumors from 4 animals in each group, and subjected to RT-PCR for murine ERα.

### Statistical Analysis

The two-tailed unpaired t test was used to determine the significance of the caspase-3 activity assay using Graph Pad software. The following symbols were used to denote statistical significance: * p<0.01, ** p<0.001.

### Results

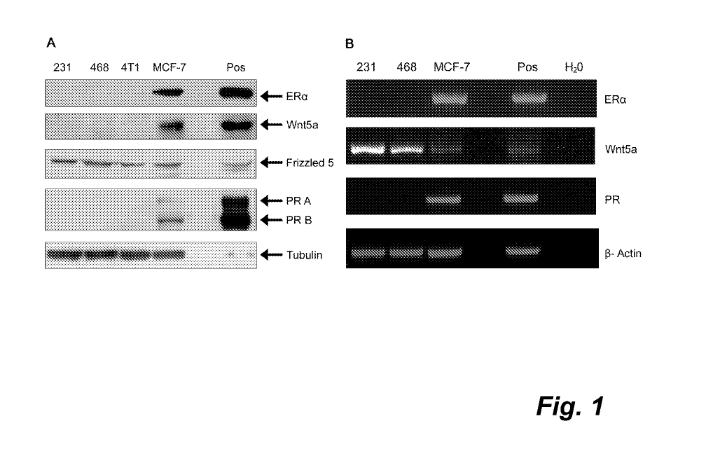

A previous study conducted on a clinical breast cancer cohort, showed that breast cancer patients that lacked expression of ERα, also lacked Wnt-5a expression (2). Therefore the experimental approach was begun by determining the endogenous expression of key proteins in three human and one mouse cell line (FIG. 1a). The human T47D breast cancer cell line was used as a positive control as it is known to express ERα, Wnt-5a, Frizzled 5 and PR (both A and β isoforms) at both the mRNA and protein level. MDA-MB-231, MDA-MB-468 and 4T1 cells lacked expression of ERα, Wnt-5a and PR, yet did they express the Wnt-5a receptor, Frizzled 5, indicating that the induction of Wnt-5a signaling is possible in these cell lines. MCF7 cells expressed all proteins tested. Next the expression of these genes was characterized at the mRNA level in the human breast cancer cells (FIG. 1b). ERα and PR mRNA was detected in MCF7 and T47D cells. Wnt-5a mRNA was detected in all cell lines, confirming previous data from the laboratory of the inventors and others suggesting that Wnt-5a expression is modified at the post-transcriptional level. This is also in concordance with clinical data from others indicating that breast cancer tumours express high levels of Wnt-5a mRNA (7).

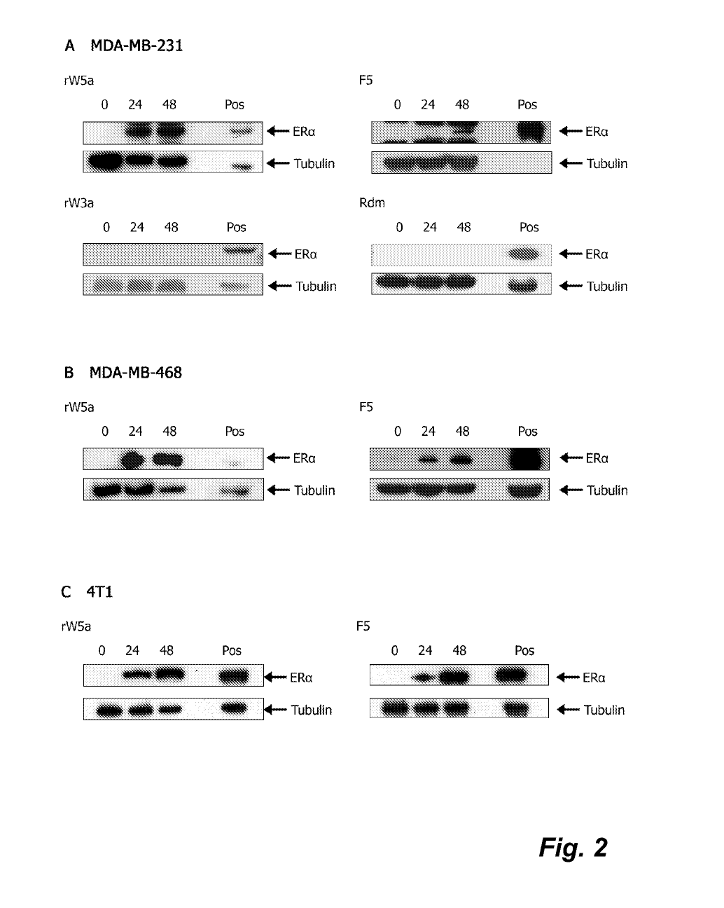

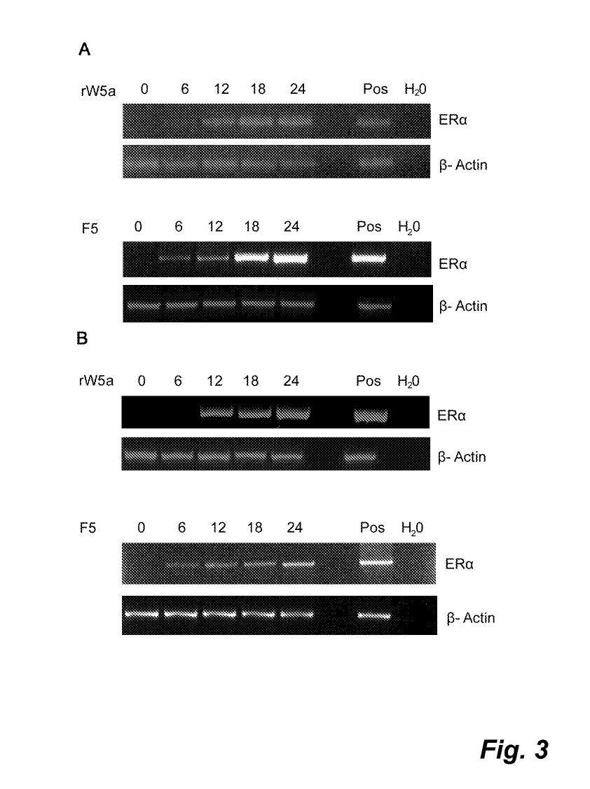

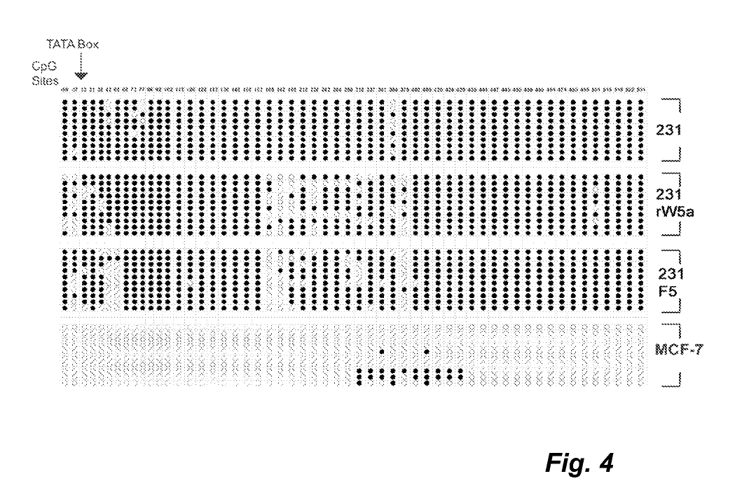

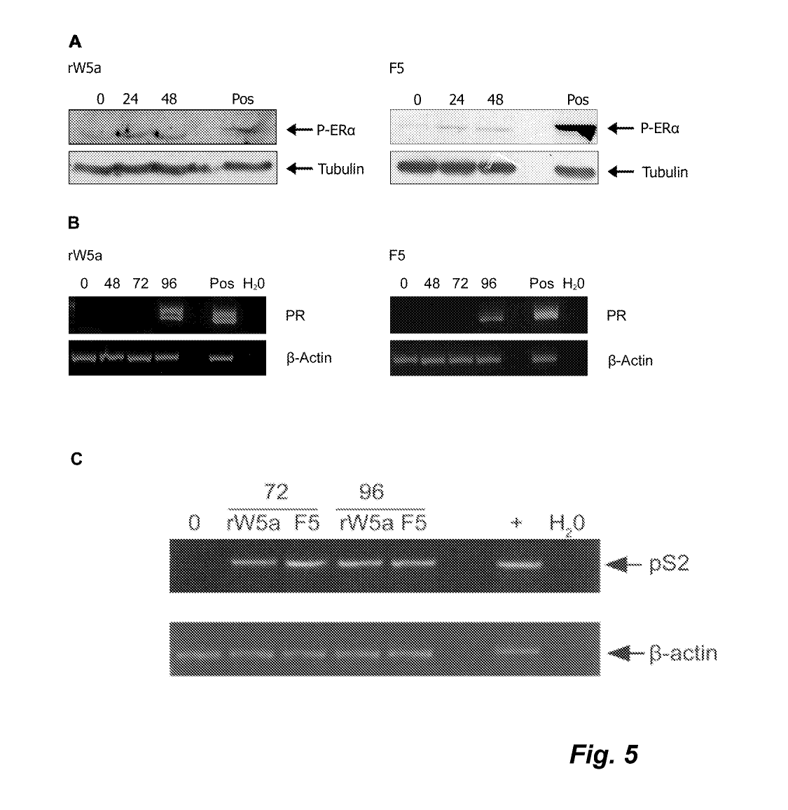

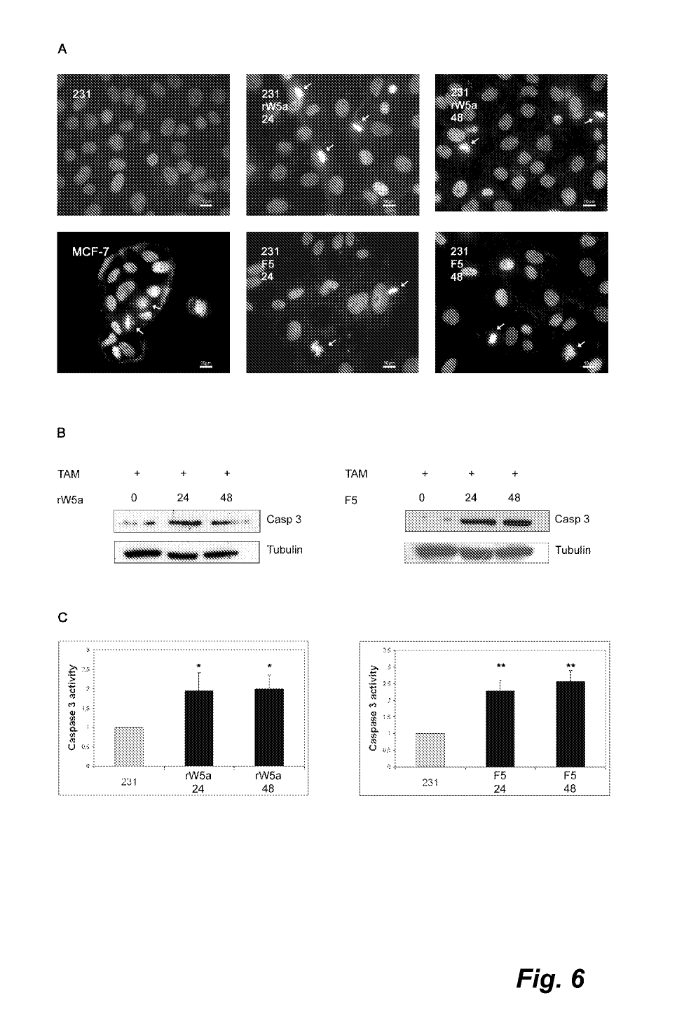

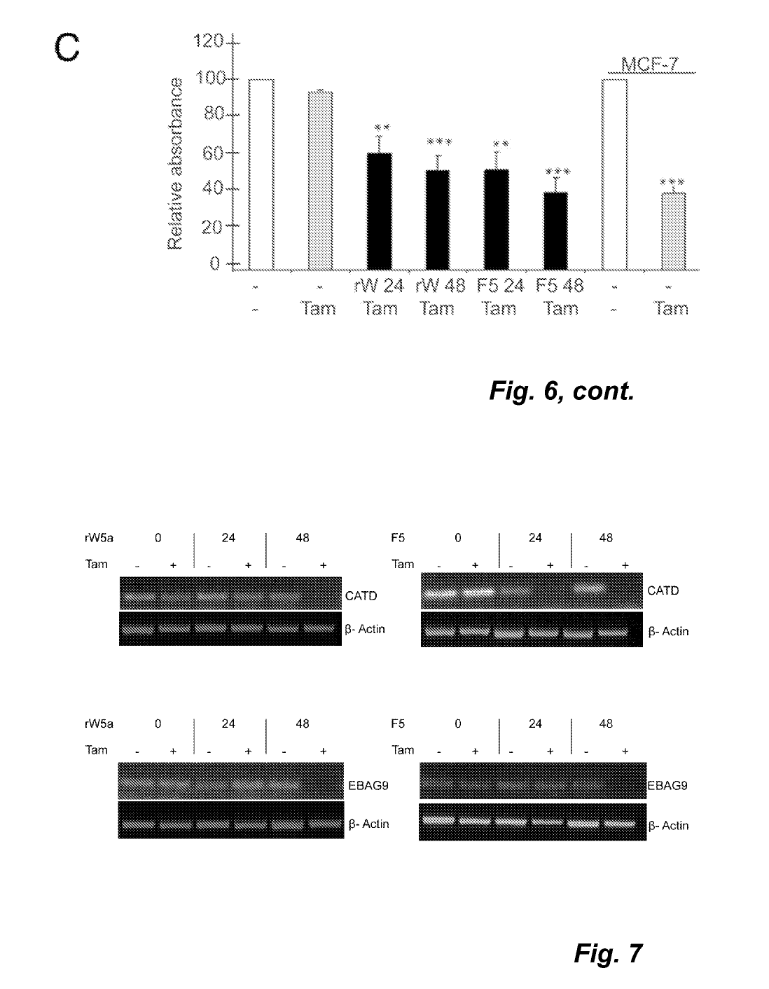

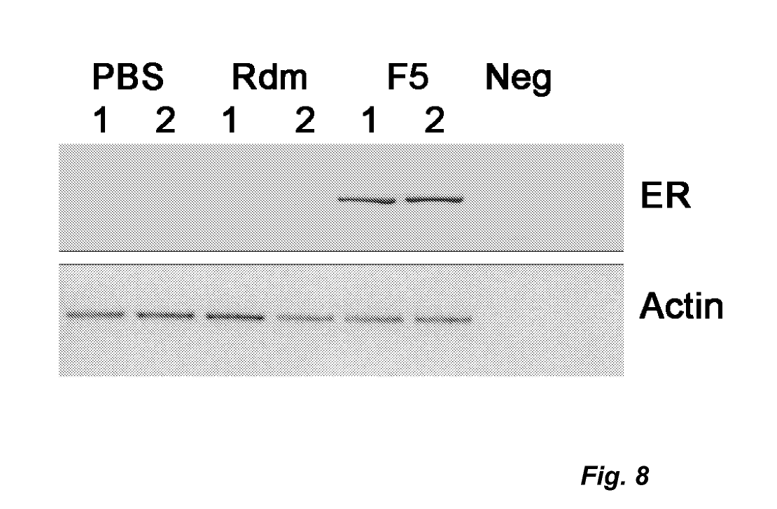

Next it was sought to determine whether restoration of Wnt-5a signaling would affect ERα expression levels in the breast cancer cell lines. MDA-MB-231 breast cancer cells were seeded onto 6 well plates in normal media, and stimulated with recombinant Wnt-5a protein for 24 and 48 hours. The ERα positive breast cancer cell lines were used as a positive control in this set of experiments, mainly to determine the correct band representing ERα on the Western Blot, rather than as a standard expression to be compared with. An increase in levels of ERα protein was observed after 24 hours (FIG. 2a, top left panel). Next it was investigated whether the Wnt-5a derived peptide developed in the laboratory, Foxy-5, would also be able to upregulate ERα expression.

This proved to be the case (FIG. 2a, top right panel). Then the specificity of this effect was investigated by stimulating the cells with recombinant Wnt-3a protein and a formylated random hexapeptide. Neither of these stimulations resulted in increased levels of ERα (FIG. 2a, bottom panels). The recombinant Wnt-5a and Foxy-5 stimulations were then repeated in two other ERα negative breast cancer cell lines. ERα levels were upregulated in both the MDA-MB-468 (FIG. 2b) and 4T1 (FIG. 2c) breast cancer cell lines following stimulation with either recombinant Wnt-5a or Foxy-5 for 24 and 48 hours.

To determine whether this ERα upregulation occurred at a transcriptional or translational level, it was investigated whether ERα mRNA upregulation occurred at time points earlier than 24 hours, when ERα protein was first detected. The human breast cancer cell lines, MDA-MB-231 and MDA-MB-468 were stimulated with recombinant Wnt-5a or Foxy-5 for 6, 12, 18 and 24 hours in order to determine at what time point ERα mRNA would be detectable. ERα mRNA was detected after 12 hours of recombinant Wnt-5a stimulation and after 6 hours of Foxy-5 stimulation in MDA-MB-231 and MDA-MB-468 cells (FIG. 3).

We then thoroughly analyzed the methylation pattern using bisufite genomic sequencing (BGS) across the ERα CpG island (FIG. 4). This analysis allowed us to clearly demonstrate that specific regions of the CpG island were demethylated in MDA-MB-231 cells which were stimulated with either rWnt-5a or Foxy 5 (FIG. 4). The same region was compared to untreated MCF-7 cells, an ERα positive breast cancer cell line. There were two main regions of demethylation following the initiation of Wnt-5a signaling, one of them being close to the TATA box and the transcription start site (10). In particular there was dramatic demethylation at positions+42, +65, +165, +192, +195, +375, relative to the transcription start site, similar to that seen in studies using HDAC and DNMT inhibitors (3, 4).

Next it was sought to determine if the upregulated ERα was functionally active. The MDA-MB-231 cell line was utilized for these experiments, and this was investigated in a number of ways. First, recombinant Wnt-5a and Foxy-5 stimulated lysates were tested for the presence of phosphorylated ERα. ERα is phosphorylated at a number of sites. It was chosen to investigate the site at serine 118, as phosphorylation at this site is most frequently used as an indicator of ERα activity. Phosphorylated ERα was detected in cells stimulated with either recombinant Wnt-5a or Foxy-5 (FIG. 45). The progesterone receptor is a downstream transcriptional target indicative of an active ERα. Therefore its transcription was investigated, following stimulation with recombinant Wnt-5a and Foxy-5 for up to 96 hours. PR mRNA was detected after 96 hours stimulation with either recombinant Wnt-5a or Foxy-5 (FIG. 5b).

Once it had been established that recombinant Wnt-5a and Foxy-5 upregulated ERα and it was indeed active and capable of downstream signaling, the clinical relevance of the data in ERα negative breast cancer cells was explored which cells are normally unresponsive to the selective estrogen receptor modulator, using tamoxifen (1, 4). Cells were stimulated or not with recombinant Wnt-5a and Foxy-5 for 24 and 48 hours, and tamoxifen was added for the final 20 hours of the experiment. The mode of action of tamoxifen has not yet been completely elucidated, however it is known that treatment of ERα positive breast epithelial cells, results in apoptosis which can be assessed visually or via analysis of key proteins in the apoptotic pathway (3). First, Hoechst staining was performed and followed by directly observed apoptotic cells displaying altered nuclear morphology (observed as chromatin condensation and fragmentation) in response to tamoxifen following stimulation with recombinant Wnt-5a or Foxy-5 for 24 and 48 hours (FIG. 6a). The fact that the majority of apoptotic cells detach makes this assay sub-optimal since it underestimates the real effect of tamoxifen on apoptosis. In order to improve the sensitivity of the assay and to determine whether this apoptosis occurred via the caspase pathway the inventors then investigated lysates from cells stimulated with either vehicle alone, or recombinant Wnt-5a or Foxy-5 and tamoxifen for the expression of cleaved caspase 3. The inventors detected higher levels of cleaved caspase-3 in Wnt-5a signalling cells than in cells treated only with tamoxifen (FIG. 6b). To quantitate these effects the inventors then investigated the activity of caspase-3 via a fluorometric assay (FIG. 6c). Stimulation of cells with recombinant Wnt-5a and tamoxifen increased the degree of apoptosis two fold, and stimulation with Foxy-5 and tamoxifen increased the degree of apoptosis almost three fold when compared to untreated cells or cells treated with tamoxifen alone. We did not include MCF-7 cells in the caspase experiments, as they have been reported not to express caspase-3. This test allowed us to clearly observe the reproducible increase in cells driven to the apoptotic pathway following the induction of Wnt-5a signaling and tamoxifen treatment. As successful tamoxifen treatment is also known to result in cell growth inhibition, we further analyzed our cells using a MTT proliferation assay (FIG. 6D). Cells stimulated with either rWnt-5a or Foxy-5 and then treated with tamoxifen, showed a statistically significant growth inhibition when compared with cells treated with tamoxifen alone (FIG. 6D). Neither rWnt-5a nor Foxy-5 had an effect on breast cancer cell proliferation alone. The effects of tamoxifen on MDA-MB-231 cells treated with rWnt-5a or Foxy-5 were very similar to the tamoxifen induced effect seen in the ERα positive MCF-7 cell line (FIG. 6D).

Next the mRNA levels of the ER-binding fragment associated antigen 9 (EBAG9) and Cathepsin D (CATD) genes were analyzed. Both of these genes are referred to as human estrogen responsive genes, as they are regulated through direct ERα binding, and their mRNA expression is indicative of an active estrogen receptor. Previous experiments indicated that ERα protein is upregulated after 24 to 48 hours stimulation with recombinant Wnt-5a or Foxy-5. Therefore MDA-MB-231 cells were grown in hormone free media and stimulated with the ligand estradiol, after sufficient time had elapsed for the ERα to be upregulated, allowing transcription of downstream targets, EBAG9 and Cathepsin D. The addition of tamoxifen for the final 20 hours of growth interfered with the binding of the ligand to the receptor, and the subsequent binding of the complex to the EREs (estrogen response elements) of the down-stream targets, and therefore expression of these genes was no longer observed at the 48 hour time point. Estradiol induced expression of CATD and EBAG9 was consistently lost at 48 hours when samples were simultaneously treated with tamoxifen, following pretreatment with either recombinant Wnt-5a or Foxy-5 (FIG. 7). In some cases expression of Cathepsin D (CATD) was also lost at the 24 hour time point, however this varied in repeated experiments. This result indicates that restoration of Wnt-5a signaling in ERα negative breast cancer cells not only upregulated ERα expression and activity, but also tamoxifen dependent repression of ERα target genes.

In view of the potential benefit of Foxy-5 for breast cancer patients, the inventors made an in vivo study into the effects of Foxy-5 mediated reconstitution of Wnt-5a signaling in a murine metastatic breast cancer model (9). They investigated the primary breast tumors from one series of animal experiments from that study, to determine whether Foxy-5 could upregulate ERα in vivo. Balb/C mice inoculated with rapidly metastic ERα negative 4T1 cells into their mammary fat pads, were treated with either PBS alone, the random control peptide (Rdm), or Foxy-5 every fourth day for 25 days. Tumors from animals treated with Foxy-5 showed strong ERα expression (FIG. 8), as opposed to tumors from animals treated with PBS alone or the Rdm control peptide. This experiment clearly shows that Foxy-5 may upregulate ERα in vivo in ERα negative breast cancer.

### DISCUSSION

The major drug used to treat breast cancer, tamoxifen, primarily mediates its effects through ERα. Expression of ERα is strongly associated with clinical response to endocrine therapy. ERα negative breast cancers are not only insensitive to tamoxifen, but also more aggressive and have a poor overall prognosis. Hence, new therapies targeting this group of patients are crucial. In this paper, the inventors report for the first time that the engagement of a natural cell surface receptor on breast epithelial cells restores the expression of ERα in ERα negative breast cancer cells. This has high clinical importance in regards to the future treatment of ERα negative breast cancer patients.

Previous research from our laboratory identified an association between ERα status and the expression of Wnt-5a in a clinical breast cancer cohort. Loss of Wnt-5a expression was shown to be significantly associated with higher histological grade of breast tumours, and with the absence of ERα (2). Here the inventors report that stimulation of three different ERα negative breast cancer cell lines, with either recombinant Wnt-5a protein or the Wnt-5a derived Foxy-5 peptide, resulted in increased ERα expression.

It is currently appreciated that the lack of expression of ERα in human breast cancer is most often due to methylation of the ERα promoter (8). The MDA-MB-231 cell line lacking ERα and Wnt-5a expression that was used in this study has been described as having a silenced ERα due to such methylation of CpG islands in the promoter region. Others have attempted to reconstitute ERα signaling in these cells via the addition of HDAC and DNMT inhibitors, and have produced similar results to that which the inventors observe by triggering Wnt-5a signaling (3). Our findings are consistent with the idea that Wnt-5a signaling acts to demethylate the ER promoter in ERα negative cells, although the epigenetic mechanisms behind this Wnt-5a induced ERα upregulation remain to be investigated.

The upregulated ERα was also phosphorylated on the Ser-118 residue and able to induce transcription of the progesterone receptor, indicative of an active and signaling ERα. The novel finding that both recombinant Wnt-5a and Foxy-5 were able to restore functional ERα lead us to investigate whether this was clinically relevant by performing functional assays utilizing the selective estrogen receptor modulator, tamoxifen. ERα negative breast cancer cells were stimulated with recombinant Wnt-5a, Foxy-5 or left unstimulated, then the ERα ligand estradiol was added and finally tamoxifen, in order to determine whether Wnt-5a signaling would render previously unresponsive cells sensitive to tamoxifen treatment. This was assessed in four ways. Firstly apoptotic cells were directly observed via Hoechst staining. Secondly increased expression of the apoptotic protein caspase-3, which is cleaved upon the induction of apoptosis, was observed via Western Blot. This was confirmed quantitatively using a fluorometric caspase 3 activity assay. Lastly the tamoxifen induced repression of downstream target genes of ERα was observed. All functional assays reported the same trend of increased apoptosis following stimulation with recombinant Wnt-5a or Foxy-5 and tamoxifen; however the capsase-3 activity assay showed the most dramatic increase. This is likely due to the design of the assay, which measures dying cells in the supernatant as well as adherent cells.

The Wnt-5a derived Foxy-5 formylated hexapeptide developed in our laboratory was able to regulate ERα expression to the same extent as recombinant Wnt-5a in the experiments. This peptide has clear clinical potential, as it possesses numerous advantages for patient use over recombinant Wnt-5a protein. Administering Wnt-5a directly to breast cancer patients is unlikely to be successful, since Wnt-5a has a specific domain that binds to cell surface heparan sulphates which significantly limits the distribution of Wnt-5a in the body. Also, Wnt-5a is a relatively large protein (43 kDa), and therefore it would be more attractive to utilise a small molecule, such as Foxy-5, which lacks the heparan sulphate-binding domain, yet can mimic the functional effects of Wnt-5a on ERα expression.

This novel approach of reconstituting ERα expression by activating a natural cell surface receptor, in order to render tumours responsive to current endocrine treatments, is of significant importance to clinical management of this common disease. Concordant treatment with a Wnt-5a mimicking hexapeptide and currently available ERα modulators may represent a novel and beneficial treatment strategy for breast cancer patients with ERα negative tumours.

### REFERENCES

1. Giacinti L, Claudio P P, Lopez M, Giordano A. Epigenetic information and estrogen receptor alpha expression in breast cancer. Oncologist 2006; 11:1-8.
2. Jonsson M, Dejmek J, Bendahl P O, Andersson T. Loss of Wnt-5a protein is associated with early relapse in invasive ductal breast carcinomas. Cancer Research 2002; 62:409-16.
3. Sharma D, Saxena N K, Davidson N E, Vertino P M. Restoration of tamoxifen sensitivity in estrogen receptor-negative breast cancer cells: tamoxifen-bound reactivated ER recruits distinctive corepressor complexes. Cancer Res 2006; 66:6370-8.
4. Sharma D, Blum J, Yang X, Beaulieu N, Macleod A R, Davidson N E. Release of methyl CpG binding proteins and histone deacetylase 1 from the Estrogen receptor alpha (ER) promoter upon reactivation in ER-negative human breast cancer cells. Mol Endocrinol 2005; 19:1740-51.
5. Bandyopadhyay A, Wang L, Chin S H, Sun L Z. Inhibition of skeletal metastasis by ectopic ERalpha expression in ERalpha-negative human breast cancer cell lines. Neoplasia 2007; 9:113-8.
6. Jang E R, Lim S J, Lee E S, et al. The histone deacetylase inhibitor trichostatin A sensitizes estrogen receptor alpha-negative breast cancer cells to tamoxifen. Oncogene 2004; 23:1724-36.
7. Lejeune S, Huguet E L, Hamby A, Poulsom R, Harris A L. Wnt5a cloning, expression, and up-regulation in human primary breast cancers. Clinical Cancer Research 1995; 1:215-22.
8. Ottaviano Y L, Issa J P, Parl F F, Smith H S, Baylin S B, Davidson N E. Methylation of the estrogen receptor gene CpG island marks loss of estrogen receptor expression in human breast cancer cells. Cancer Res 1994; 54: 2552-5.
9. Säfholm A, et al. (2008) A Wnt-5a-Derived Hexapeptide F5 Inhibits Breast Cancer Metastasis In vivo by Targeting Cell Motility. Clin Cancer Res 14:6556-6563.
10. Green S, et al. (1986) Human oestrogen receptor cDNA: sequence, expression and homology to v-erb-A. Nature 320(6058):134-139.

1. A method of treatment of a subtype of breast cancer characterized by the lack of estrogen receptor-α activity wherein a therapeutically effective amount of a Wnt5-α protein or a peptide thereof is administered to a patient in need of said treatment.

2. The method of claim 1, wherein said breast cancer is a breast cancer in an estrogen receptor-α negative patient.

3. The method of claim 1, wherein the treatment also includes an endocrine treatment.

4. The method of claim 1, wherein the treatment also includes treatment with a selective estrogen receptor modulator.

5. The method of claim 4, wherein said selective estrogen receptor modulator is tamoxifen.

6. The method of claim 1, wherein the treatment also includes treatment with an aromatase inhibitor.

7. The method of claim 1, wherein said Wnt5-α protein is a recombinant protein.

8. The method of claim 1, wherein said Wnt5-α peptide has one of the following sequences

| | |
|---|---|
| GMDGCEL | SEQ. ID. NO. 2 |
| EGMDGCEL | SEQ. ID. NO. 3 |
| SEGMDGCEL | SEQ. ID. NO. 4 |
| TSEGMDGCEL | SEQ. ID. NO. 5 |
| KTSEGMDGCEL | SEQ. ID. NO. 6 |
| NKTSEGMDGCEL | SEQ. ID. NO. 7 |
| CNKTSEGMDGCEL | SEQ. ID. NO. 8 |
| LCNKTSEGMDGCEL | SEQ. ID. NO. 9 |
| RLCNKTSEGMDGCEL | SEQ. ID. NO. 10 |
| GRLCNKTSEGMDGCEL | SEQ. ID. NO. 11 |
| QGRLCNKTSEGMDGCEL | SEQ. ID. NO. 12 |
| TQGRLCNKTSEGMDGCEL | SEQ. ID. NO. 13 |
| GTQGRLCNKTSEGMDGCEL | SEQ. ID. NO. 14 |
| LGTQGRLCNKTSEGMDGCEL | SEQ. ID. NO. 15 |

9. The method of claim 1, wherein said Wnt5-α peptide has one of the following sequences

| | |
|---|---|
| MDGCEL | SEQ. ID. NO. 1 |
| GMDGCEL | SEQ. ID. NO. 2 |
| EGMDGCEL | SEQ. ID. NO. 3 |
| SEGMDGCEL | SEQ. ID. NO. 4 |
| TSEGMDGCEL | SEQ. ID. NO. 5 |
| KTSEGMDGCEL | SEQ. ID. NO. 6 |
| NKTSEGMDGCEL | SEQ. ID. NO. 7 |
| CNKTSEGMDGCEL | SEQ. ID. NO. 8 |
| LCNKTSEGMDGCEL | SEQ. ID. NO. 9 |
| RLCNKTSEGMDGCEL | SEQ. ID. NO. 10 |
| GRLCNKTSEGMDGCEL | SEQ. ID. NO. 11 |
| QGRLCNKTSEGMDGCEL | SEQ. ID. NO. 12 |
| TQGRLCNKTSEGMDGCEL | SEQ. ID. NO. 13 |
| GTQGRLCNKTSEGMDGCEL | SEQ. ID. NO. 14 |
| LGTQGRLCNKTSEGMDGCEL | SEQ. ID. NO. 15 |

and is present as a formylated derivative thereof.

10.-18. (canceled)

19. A method for restoring estrogen receptor-α activity by administering a therapeutically active amount of Wnt5-α protein or a peptide thereof to a human lacking estrogen receptor-α for a time sufficient to induce such estrogen receptor-α activity by restoring such receptors.

20. A method for facilitating or enhancing endocrine post-treatment in a human suffering from breast cancer and lacking estrogen receptor-α activity, wherein a therapeutically effective amount of Wnt5-α protein of a peptide thereof is administered for a time sufficient to induce estrogen receptor-α activity.

21. The method of claim 20, wherein said endocrine post-treatment is treatment with a selective estrogen receptor modulator.

22. The method of claim 21, wherein the selective estrogen receptor modulator is tamoxifen.

23. The method of claim 20, wherein said endocrine post-treatment is treatment with an aromatase inhibitor.

24. The method of claim 20, wherein the Wnt5-α peptide is at least one peptide selected from the group consisting of

| | |
|---|---|
| MDGCEL | SEQ. ID. NO. 1 |
| GMDGCEL | SEQ. ID. NO. 2 |
| EGMDGCEL | SEQ. ID. NO. 3 |
| SEGMDGCEL | SEQ. ID. NO. 4 |
| TSEGMDGCEL | SEQ. ID. NO. 5 |
| KTSEGMDGCEL | SEQ. ID. NO. 6 |
| NKTSEGMDGCEL | SEQ. ID. NO. 7 |
| CNKTSEGMDGCEL | SEQ. ID. NO. 8 |
| LCNKTSEGMDGCEL | SEQ. ID. NO. 9 |
| RLCNKTSEGMDGCEL | SEQ. ID. NO. 10 |
| GRLCNKTSEGMDGCEL | SEQ. ID. NO. 11 |
| QGRLCNKTSEGMDGCEL | SEQ. ID. NO. 12 |
| TQGRLCNKTSEGMDGCEL | SEQ. ID. NO. 13 |
| GTQGRLCNKTSEGMDGCEL | SEQ. ID. NO. 14 |
| LGTQGRLCNKTSEGMDGCEL | SEQ. ID. NO. 15 |

and being present as a formylated derivative thereof.

25. The method of claim 20, wherein the Wnt5-α peptide is at least one peptide selected from the group consisting of

| | |
|---|---|
| GMDGCEL | SEQ. ID. NO. 2 |
| EGMDGCEL | SEQ. ID. NO. 3 |
| SEGMDGCEL | SEQ. ID. NO. 4 |
| TSEGMDGCEL | SEQ. ID. NO. 5 |
| KTSEGMDGCEL | SEQ. ID. NO. 6 |
| NKTSEGMDGCEL | SEQ. ID. NO. 7 |
| CNKTSEGMDGCEL | SEQ. ID. NO. 8 |
| LCNKTSEGMDGCEL | SEQ. ID. NO. 9 |
| RLCNKTSEGMDGCEL | SEQ. ID. NO. 10 |
| GRLCNKTSEGMDGCEL | SEQ. ID. NO. 11 |
| QGRLCNKTSEGMDGCEL | SEQ. ID. NO. 12 |
| TQGRLCNKTSEGMDGCEL | SEQ. ID. NO. 13 |
| GTQGRLCNKTSEGMDGCEL | SEQ. ID. NO. 14 |
| LGTQGRLCNKTSEGMDGCEL | SEQ. ID. NO. 15 |

26. The method of claim 2, wherein the treatment also includes an endocrine treatment.

27. The method of claim 8, wherein the treatment also includes an endocrine treatment.

28. The method of claim 9, wherein the treatment also includes an endocrine treatment.

29. The method of claim 2, wherein the treatment also includes treatment with a selective estrogen receptor modulator.

30. The method of claim 3, wherein the treatment also includes treatment with a selective estrogen receptor modulator.

31. The method of claim 8, wherein the treatment also includes treatment with a selective estrogen receptor modulator.

32. The method of claim 9, wherein the treatment also includes treatment with a selective estrogen receptor modulator.

33. The method of claim 29, wherein said selective estrogen receptor modulator is tamoxifen.

34. The method of claim 30, wherein said selective estrogen receptor modulator is tamoxifen.

35. The method of claim 31, wherein said selective estrogen receptor modulator is tamoxifen.

36. The method of claim 32, wherein said selective estrogen receptor modulator is tamoxifen.

37. The method of claim 2, wherein the treatment also includes treatment with an aromatase inhibitor.

38. The method of claim 3, wherein the treatment also includes treatment with an aromatase inhibitor.

39. The method of claim 8, wherein the treatment also includes treatment with an aromatase inhibitor.

40. The method of claim 9, wherein the treatment also includes treatment with an aromatase inhibitor.

41. The method of claim 2, wherein said Wnt5-α protein is a recombinant protein.

42. The method of claim 3, wherein said Wnt5-α protein is a recombinant protein.

43. The method of claim 4, wherein said Wnt5-α protein is a recombinant protein.

44. The method of claim 5, wherein said Wnt5-α protein is a recombinant protein.

45. The method of claim 6, wherein said Wnt5-α protein is a recombinant protein.


SEQUENCE LISTING (15 sequences)

SEQ ID NO: 1 - protein, 6 aa, Homo sapiens
MDGCEL

SEQ ID NO: 2 - protein, 7 aa, Homo sapiens
GMDGCEL

SEQ ID NO: 3 - protein, 8 aa, Homo sapiens
EGMDGCEL

SEQ ID NO: 4 - protein, 9 aa, Homo sapiens
SEGMDGCEL

SEQ ID NO: 5 - protein, 10 aa, Homo sapiens
TSEGMDGCEL

SEQ ID NO: 6 - protein, 11 aa, Homo sapiens
KTSEGMDGCEL

SEQ ID NO: 7 - protein, 12 aa, Homo sapiens
NKTSEGMDGCEL

SEQ ID NO: 8 - protein, 13 aa, Homo sapiens
CNKTSEGMDGCEL

SEQ ID NO: 9 - protein, 14 aa, Homo sapiens
LCNKTSEGMDGCEL

SEQ ID NO: 10 - protein, 15 aa, Homo sapiens
RLCNKTSEGMDGCEL

SEQ ID NO: 11 - protein, 16 aa, Homo sapiens
GRLCNKTSEGMDGCEL

SEQ ID NO: 12 - protein, 17 aa, Homo sapiens
QGRLCNKTSEGMDGCEL

SEQ ID NO: 13 - protein, 18 aa, Homo sapiens
TQGRLCNKTSEGMDGCEL

SEQ ID NO: 14 - protein, 19 aa, Homo sapiens
GTQGRLCNKTSEGMDGCEL

SEQ ID NO: 15 - protein, 20 aa, Homo sapiens
LGTQGRLCNKTSEGMDGCEL